(19) United States
(12) Patent Application Publication
(10) Pub. No.: US 2026/0124341 A1
(43) Pub. Date: May 7, 2026

(54) FISH LIVER DECELLULARIZED EXTRACELLULAR MATRIX BASED MICROFLUIDIC 3D PRINTING HYDROGEL, AND PREPARATION METHOD AND APPLICATION THEREOF

(71) Applicant: NANJING DRUM TOWER HOSPITAL, Nanjing (CN)

(72) Inventor: Jinglin WANG, Nanjing (CN)

(73) Assignee: NANJING DRUM TOWER HOSPITAL, Nanjing (CN)

(21) Appl. No.: 18/991,797

(22) Filed: Dec. 23, 2024

(30) Foreign Application Priority Data
Nov. 4, 2024 (CN) 2024115549091

(51) Int. Cl. A61L 27/36; A61L 27/22; A61L 27/38; A61L 27/52; B33Y 70/00

(52) U.S. Cl. CPC A61L 27/3633; A61L 27/222; A61L 27/3683; A61L 27/3687; A61L 27/3804; A61L 27/52; B33Y 70/00; A61L 2300/64; A61L 2430/28; A61L 2430/40

(57) ABSTRACT

A fish liver decellularized extracellular matrix based microfluidic 3D printing hydrogel for liver regeneration, and a preparation method thereof are provided. A fish liver decellularized extracellular matrix (dECM) is combined with gelatin methacryloyl (GelMA), and loaded with hepatic spheroids derived from induced pluripotent stem cells (iPSC-hep) for liver regeneration. The fish liver decellularized extracellular matrix based microfluidic 3D printing hydrogel of the present disclosure has excellent biocompatibility and retains intact endogenous growth factors, maintains the biological activity of cells, ensures effective cell encapsulation, and is conducive to robust functional expression of iPSC-hep. After being transplanted in vivo, the hydrogel significantly improves the survival rate and liver function of mice with acute liver failure, and promotes liver regeneration and repair.

### CROSS REFERENCE TO THE RELATED APPLICATIONS

This application is based upon and claims priority to Chinese Patent Application No. 2024115549091, filed on Nov. 4, 2024, the entire contents of which are incorporated herein by reference.

### TECHNICAL FIELD

The present disclosure belongs to the field of biomedical materials, and specifically relates to a fish liver decellularized extracellular matrix based microfluidic 3D printing hydrogel for liver regeneration and a preparation method thereof.

### BACKGROUND

Liver transplantation is an efficient method for treating end-stage liver diseases, but the scarcity of donor livers limits the clinical application of liver transplantation. Tissue engineering is considered as a promising alternative to donor livers due to an ability to construct functional hepatic tissues. Hydrogels are an advanced material for shaping various structures required for liver transplantation, and have good biocompatibility and cell proliferation ability for hepatic spheroids derived from induced pluripotent stem cells (iPSC-heps). However, hydrogels usually exhibit limited biological activity, and are significantly different from natural hepatic tissues in composition. In addition, most hydrogels require complex manufacturing processes, causing potential biosafety problems and reducing therapeutic effects. Therefore, the demand for innovative hydrogels that have inherent biological activity and provide sufficient support for liver function has not been met.

As a natural material, decellularized fish liver has excellent biocompatibility and retains intact endogenous growth factors, including collagen and glycosaminoglycans. Compared with terrestrial animal liver, fish liver resources from aquatic products are abundant and cost-effective. In addition, microfluidics technology is recognized for precise control of fluid dynamics, and is an advanced form used for additive manufacturing in drug delivery, cell culture, biosensors, and the like. 3D printing allows personalized design of implantable hydrogels to meet specific clinical needs. With these advantages, the combination of microfluidics and 3D printing technology makes it possible to produce customizable hydrogels for liver regeneration. Therefore, it is very desirable to combine a fish liver decellularized hydrogel as a biological chain with a microfluidic assisted 3D printing technology to develop a new scaffold that can effectively promote liver repair.

### SUMMARY

Objective of the disclosure: In order to solve the above technical problems, the present disclosure proves a simple microfluidic 3D printing technology to prepare a fish liver decellularized extracellular matrix based hydrogel for liver regeneration, which is used for liver repair by orthotopic transplantation. The hydrogel is prepared by a method using the microfluidic 3D printing technology, which is simple, versatile and convenient for large-scale production.

The technical solution: A fish liver decellularized extracellular matrix based microfluidic 3D printing hydrogel, wherein a fish liver decellularized extracellular matrix is combined with gelatin methacryloyl as a scaffold, hepatic spheroids derived from human induced pluripotent stem cells are used as a cell source, and a microfluidic 3D printing technology is used to prepare a mixed material hydrogel scaffold.

The preparation steps of a fish liver decellularized extracellular matrix based microfluidic 3D printing hydrogel are as follows:
1) preparing hepatic spheroids derived from human induced pluripotent stem cells;
2) preparing a fish liver decellularized extracellular matrix; and
3) designing a 3D scaffold model; fully mixing gelatin methacryloyl, the fish liver decellularized extracellular matrix prepared in step 2), the hepatic spheroids derived from human induced pluripotent stem cells prepared in step 1), and a photoinitiator to form a biological link; and solidifying the biological link under ultra violet (UV) irradiation into a scaffold with a fixed shape.

The specific steps of step 2) are as follows:
a. extracting the liver and intact blood vessels from a fresh fish;
b. continuously perfusing the liver with a surfactant solution at room temperature for decellularization;
c. washing the material obtained from step b with phosphate buffered saline; and
d. dissolving the fish liver decellularized extracellular matrix obtained from step c in an acetic acid aqueous solution containing pepsin.

Preferably, wherein the photoinitiator is 2-hydroxy-2-methyl-1phenyl-1acetone.

Preferably, wherein the surfactant is sodium dodecyl sulfonate or sodium deoxycholate.

Preferably, wherein the concentration of the surfactant solution used in step b is 1%(g/100 mL, i.e., 1 g of a surfactant dissolved in 100 mL of water), the continuous perfusion rate for the fish liver is 10 mL/min and the perfusion time is 3 h.

Preferably, wherein the mass concentration of pepsin in the acetic acid aqueous solution of pepsin in step d is 1% (g/100 mL, i.e., 1 g of a surfactant dissolved in 100 mL of water), and the concentration of acetic acid is 100 mM.

Preferably, wherein the mass ratio of the fish liver decellularized extracellular matrix to the gelatin methacryloyl to the photoinitiator in the reaction system is (1.5-3): 7.5:1.

The present disclosure further provides an application of the fish liver decellularized extracellular matrix based microfluidic 3D printing hydrogel in preparation of drugs for liver regeneration.

Beneficial effects:
(1) The present disclosure designs the fish liver decellularized extracellular matrix based microfluidic 3D printing hydrogel for liver regeneration, which has excellent biocompatibility.
(2) The fish liver decellularized extracellular matrix based hydrogel for liver regeneration provided by the present disclosure is prepared by the method using the microfluidic 3D printing technology, which is simple, convenient to operate and easy for large-scale preparation, and has high repeatability, low technical requirements, high versatility, and high flexibility.
(3) The fish liver decellularized extracellular matrix based microfluidic 3D printing hydrogel prepared by the present disclosure for liver regeneration retains a unique fiber structure supporting cell adhesion and migration, maintains the biological activity of cells, ensures effective cell encapsulation, and is conducive to robust functional expression of iPSC-hep. After being transplanted in vivo, the hydrogel significantly improves the survival rate and liver function of mice with acute liver failure, and promotes liver regeneration and repair.

### BRIEF DESCRIPTION OF THE DRAWINGS

FIGS. 1A-1E are schematic diagrams illustrating preparation and evaluation of a fish liver decellularized extracellular matrix (dECM), where FIG. 1A shows the effect of 1% SDS on decellularization of fish liver for 15, 30, 120, 240, and 360 min respectively, with a scale of 0.5 cm; FIG. 1B shows the DNA content in native and decellularized fish livers; FIG. 1C shows scanning electron microscopy, H&E and DAPI staining images of native and decellularized fish livers, with scales of 100 μm (i), 20 μm (ii, iii, iv), and 50 μm (v, vi) respectively; FIG. 1D shows immunofluorescence staining of laminin, collagen I and collagen IV in native and decellularized fish livers, cell nuclei being displayed by DAPI staining, with a scale of 100 μm; and FIG. 1E shows DAPI quantitative fluorescence analysis of group D, demonstrating the clearance of cell nuclei after decellularization.

FIGS. 2A-2G are schematic diagrams illustrating dECM hydrogel based bioprinting characterization, where FIG. 2A shows preparation of a dECM and GelMA mixed microfluidic 3D printing hydrogel; FIG. 2B is a schematic diagram of the second, fourth, and sixth layers of scaffolds by microfluidic 3D bioprinting; FIGS. 2C-2D show the fluorescence macroscopic images (FIG. 2C) and close-up images (FIG. 2D) of the scaffolds with green fluorescent nanoparticles added to the second, fourth, and sixth layers; FIG. 2E is an SEM image depicting the microstructure inside a bioprinted scaffold, with scales of 100 μm (i) and 50 μm (ii); and FIGS. 2F-2G show comparative analysis diagrams illustrating the swelling ratio (FIG. 2F) and degradation rate (FIG. 2G) of a dECM-free bioink group (GelMA), a 1.5% dECM bioink group, and a 3% dECM bioink group.

FIGS. 3A-3F are schematic diagrams illustrating evaluation of biocompatibility and maintenance of cell function of a dECM based hydrogel scaffold, where FIG. 3A shows imaging of live and dead cells on days 1, 3, 5 and 7, after iPSC-heps are seeded on scaffolds printed with GelMA, the 1.5% dECM hydrogel and the 3% dECM hydrogel, with a scale of 50 μm; FIG. 3B is a schematic diagram illustrating colonization of cells cultured on the dECM based hydrogel scaffold for 7 days under a scanning electron microscope, with scales of 200 μm, 100 μm, and 20 μm from top to bottom respectively; FIG. 3C is a quantitative analysis diagram of staining of live and dead cells cultured on three scaffolds for 7 days; FIG. 3D shows mRNA expression levels on the 1.5% dECM based hydrogel and 3% dECM based hydrogel scaffolds; and FIGS. 3E-3F show immunofluorescence staining of cells on the 3% dECM hydrogel scaffold, with a scale of 10 μm.

FIGS. 4A-4H are schematic diagrams illustrating efficacy evaluation of transplantation with the dECM based hydrogel scaffold in treatment of ALF mice, where FIG. 4A is a schematic diagram illustrating an ALF mouse model and transplantation of a hydrogel scaffold in a liver; FIG. 4B is a schematic diagram illustrating the hydrogel scaffold adhered to the liver; FIG. 4C is a schematic diagram illustrating colonization of the hydrogel scaffold on the liver by in vivo imaging of a small animal; FIGS. 4D-4F are schematic diagrams showing the survival rate (FIG. 4D), aspartate aminotransferase (AST) (FIG. 4E), and alanine aminotransferase (ALT) (FIG. 4F) levels of four groups of mice; and FIGS. 4G-4H show H&E staining (FIG. 4H), and the degree of liver damage and corresponding Suzuki scores (FIG. 4G), with a scale of 100 μm in (FIG. 4H).

FIGS. 5A-5H are schematic diagrams illustrating immunohistochemical evaluation of the therapeutic effect of transplantation with the dECM based hydrogel scaffold, where FIGS. 5A-5C show merged fluorescence images of representative Ki-67 (FIG. 5A), TUNEL (FIG. 5B), and CD68, Nrf2, HO-1, and DAPI (FIG. 5C) of different groups of liver samples; and FIGS. 5D-5H show fluorescence quantitative analysis diagrams of Ki-67 (FIG. 5D), TUNEL (FIG. 5E), CD68 (FIG. 5F), Nrf2 (FIG. 5G), and HO-1 (FIG. 5H), with a scale of 100 μm.

### DETAILED DESCRIPTION OF THE EMBODIMENTS

For better understanding of the present disclosure, the present disclosure will be further described in conjunction with embodiments and accompanying drawings. The embodiments are merely used to explain the present disclosure and do not constitute a limitation on the scope of protection of the present disclosure.

The human induced pluripotent stem cells (hiPSC) used in the following embodiments were purchased from the stem cell bank of the Chinese Academy of Sciences.

The meaning of concentration in the following embodiments refers to the mass of solute in 100 mL of solvent. For example, the concentration of GelMA in a reaction system being 7.5% means that the mass of GelMA in every 100 mL of reaction solvent is 7.5 g.

### Embodiment 1

### Preparation of Fish Liver Decellularized Extracellular Matrix Based Microfluidic 3D Printing Hydrogel

(1) Preparation of hepatic spheroids derived from human induced pluripotent stem cells (hiPSC-hep) (preparation steps cited in existing technical literature, Chen, Sitong et al. Hepatic spheroids derived from human induced pluripotent stem cells in bio-artificial liver rescue porcine acute liver failure. Cell Research, (2019)0:1-3):

Human induced pluripotent stem cells (hiPSC) were cultured in mTeSR1 medium containing a matrix gel at 37° C. with 5% CO2 in a 6-well plate (5×105 cells per well). Then, the cells were cultured in RPMI1640 medium containing Activin a, BMP4, bFGF, B27, and Wnt3a for 1 day, and then transferred to RPMI1640 medium containing Activin a, BMP4, and bFGF for 3 days to stimulate the final development of endodermal cells. To promote the formation of hepatic cells, the endodermal cells were cultured in RPMI1640 medium containing KGF, SB431542, and B27 for 2 days, and then cultured in RPMI1640 medium containing KGF, BMP4, BMP2, bFGF, and B27 for 3 days. To promote the differentiation of hepatic cells into hepatic progenitor cells (HPCs), hepatic mother cells were cultured in DMEM/F12 medium containing B27, forskolin, SB431542 EGF, CHIR99021, LPA, Dex, and S1P for 6-8 days. To generate mature hepatic spheroids (hiPSC-Heps), HPCs were cultured in Williams'E medium containing B27, forskolin, and SB431542 for 21 days.

(2) Preparation of fish liver decellularized extracellular matrix: Intact liver and blood vessels were extracted from a fresh fish. Then, the liver was continuously perfused with sodium dodecyl sulfate (SDS) at room temperature for decellularization. Subsequently, the liver was washed with phosphate buffered saline to eliminate any residual decellularizing agent. All the above operations were performed under sterile conditions. The prepared fish liver decellularized extracellular matrix (dECM) was dissolved in a solution containing pepsin and acetic acid. The concentration of SDS was 1%, the concentration of pepsin was 1%, and the concentration of acetic acid was 100 mM. The continuous perfusion rate was 10 mL/min and the perfusion time was 3 h.

(3) Preparation of fish liver decellularized extracellular matrix based microfluidic 3D printing hydrogel

After a microfluidic printing device was disinfected, a 3D scaffold model was designed for printing. The fish liver decellularized extracellular matrix prepared in step (2), GelMA, the hiPSC-heps prepared in step (1) and a photoinitiator HMPP (2-hydroxy-2-methyl-1phenyl-1acetone) were fully mixed in water to form a biological link, which was immediately solidified under ultraviolet radiation into a hydrogel scaffold with a fixed shape. In the reaction system, the concentrations of the dECM were 1.5% and 3% respectively, the concentration of the GelMA was 7.5%, the concentration of the HMPP was 1%, and the amount of the hiPSC-heps used was in 107 cells.

### Embodiment 2

Evaluation of Fish Liver Decellularized Extracellular Matrix (dECM)

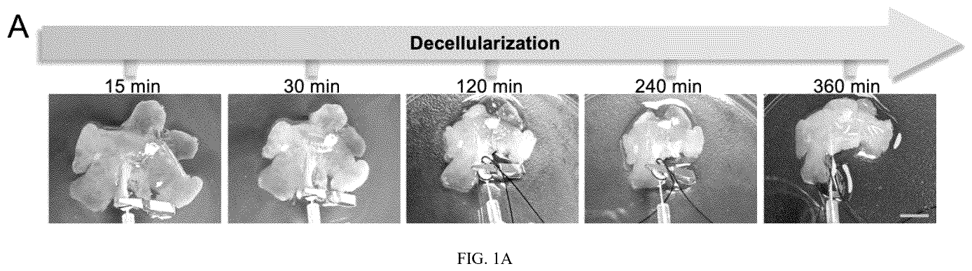

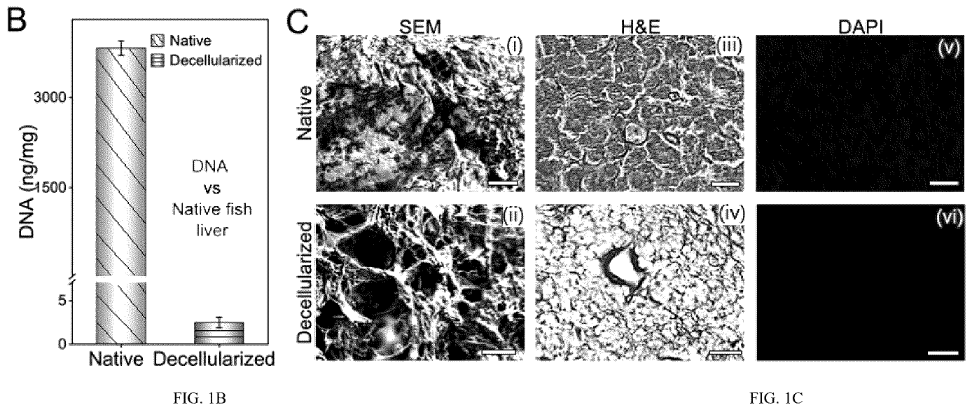

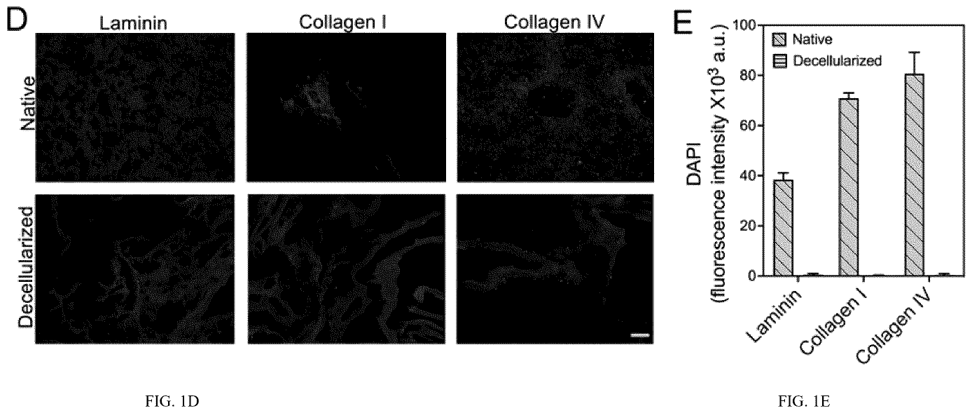

Using 1% sodium dodecyl sulfate (SDS) as a decellularizing agent, the decellularization process was accelerated through portal vein circulation of the fish liver. Over the perfusion time, the overall color of the liver transitioned from opaque to transparent, progressing from the center to the periphery, and the integrity of the intrahepatic duct system and overall structure was fully retained (FIG. 1A). As shown in FIG. 1B, DNA quantitative analysis showed that less than 50 ng of double stranded DNA was present per milligram of tissues, indicating that the decellularization process effectively eluted DNA from the fresh tissues. Compared with the native fish liver, scanning electron microscopy (SEM) showed that even after decellularization, a unique fiber structure supporting cell adhesion and migration was retained (FIG. 1C). In addition, hematoxylin and eosin (H&E) staining and 4′,6-diamino-2-phenyl indole (DAPI) immunofluorescence staining showed that no obvious residual nuclei were present in the decellularized hepatic tissues, confirming effective removal of cells from the liver. It could be observed by H&E staining that although the structure of hepatic cells was dissolved, the internal system of the liver remained intact, which meant that the essential basic ECM and structural scaffold for liver function were preserved, which was crucial for maintaining the structure and function of organs. On this basis, key components of the ECM involved in tissue regeneration were stained, and the research results showed that components such as laminin, collagen I, and collagen IV were retained after decellularization (FIG. 1D). In addition, although DAPI quantification confirmed the removal of the cell nuclei (FIG. 1E), the hepatic duct structure remained intact, indicating that the decellularization process effectively retained the vascular and biliary structures of the liver, which was crucial for subsequent recellularization and functional recovery.

### Embodiment 3

dECM Hydrogel Based Bioprinting Characterization

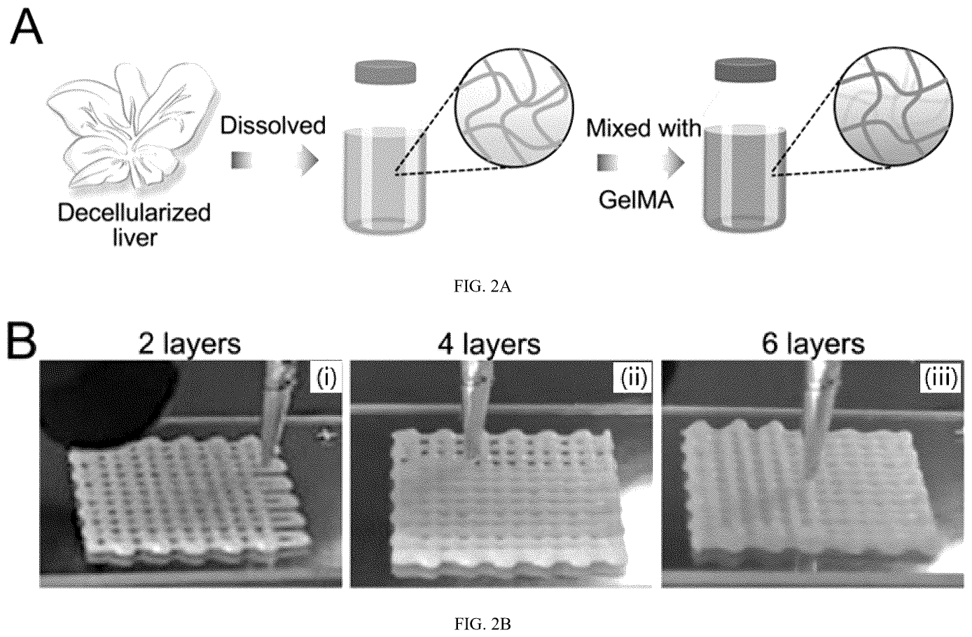

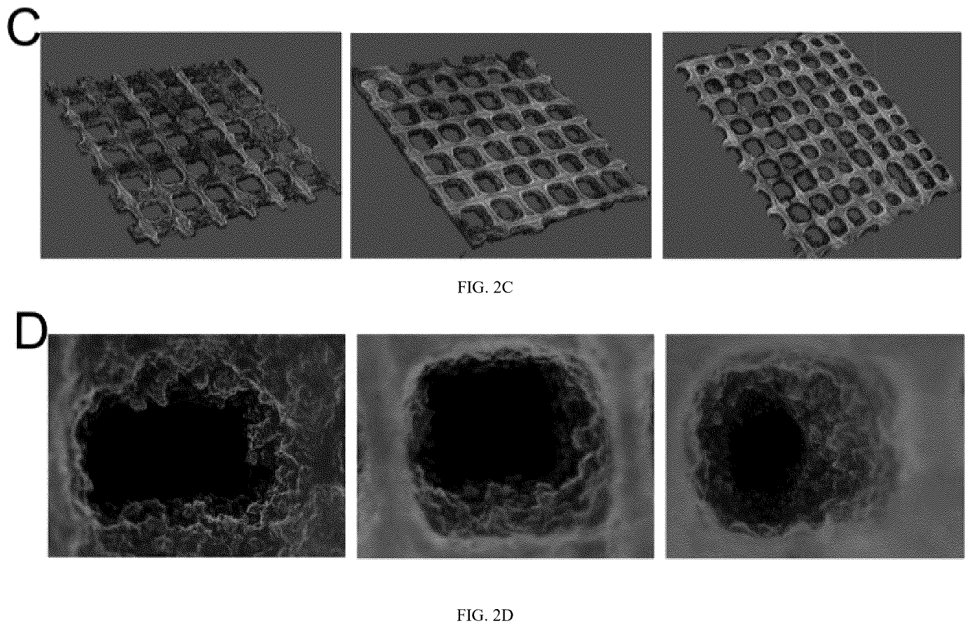

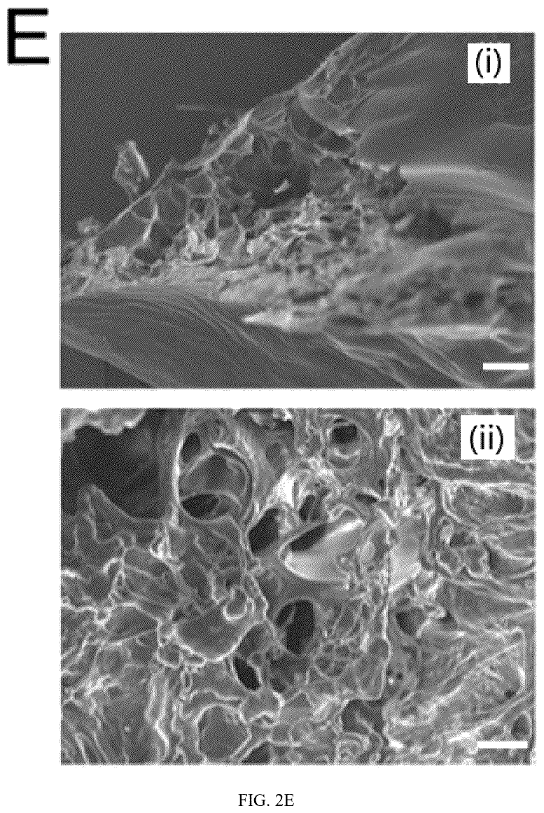

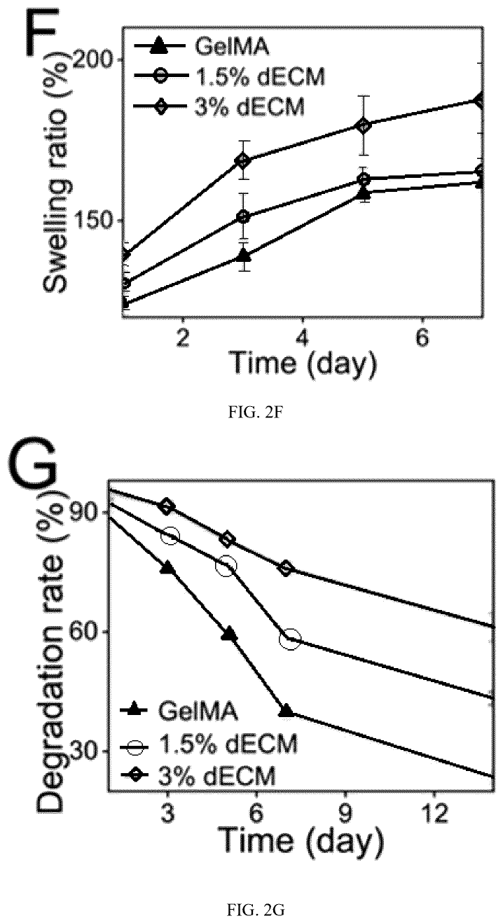

To use the dECM as a hydrogel scaffold for tissue engineering, pepsin and acetic acid were used for further digestion. However, the obtained solution had a low viscosity and was unsuitable for use as a biological link. To solve this problem, addition of GelMA, a hydrogel extracted from gelatin, which is famous for excellent biocompatibility, was taken into account, mainly because GelMA can support the adhesion and proliferation of various cell types. In addition, the performance of GelMA can be well adjusted to match the mechanical properties of different tissues and provide appropriate adjustable viscosity, which is beneficial for a bioprinting process. Therefore, the dECM was mixed with GelMA to produce a photocurable gel ink suitable for bioprinting (FIG. 2A). 3D printing performed under operation of a microfluidic device showed that the bioink was smoothly extruded from a nozzle and rapidly solidified under ultraviolet irradiation into a scaffold with a fixed shape (FIG. 2B). Green fluorescent nanoparticles were added to the bioink, and it could be seen that the dECM based hydrogel was printed into 2, 4 and 6 layers. The printing structure remained intact, and the thickness increased with layers (FIGS. 2C-2D). It was indicated that the biological link had good photo-curing performance and an ability to form a multi-layer structural scaffold. Scanning electron microscopy showed a large number of interconnected internal pores, which facilitated cell adhesion and proliferation (FIG. 2E). Subsequently, the water absorption capacity of the dECM based hydrogels of different concentrations was tested. The hydrogel was soaked in phosphate buffered saline (PBS), and the total weight gradually increased over time, indicating that the scaffold had hydrophilicity and water absorption capacity (FIG. 2F). It was indicated that the scaffold could effectively absorb surrounding medium and nutrients during cell culture and tissue regeneration to provide a favorable fluid environment for cell proliferation. Then, the dECM hydrogels of different concentrations were placed in PBS for 14 days to evaluate the stability of the scaffold. As shown in FIG. 2G, the 3% dECM hydrogel effectively maintained the structure and demonstrated sufficient stability in cell culture applications.

### Embodiment 4

### Evaluation of Biocompatibility and Maintenance of Cell Function of Decm Based Hydrogel Scaffold

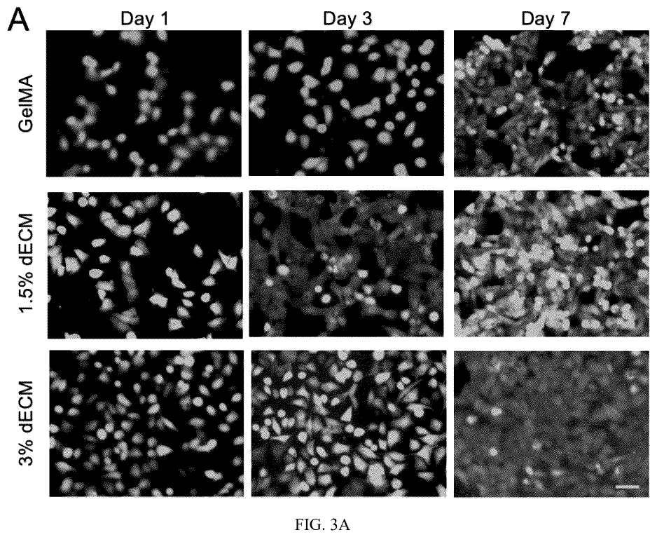

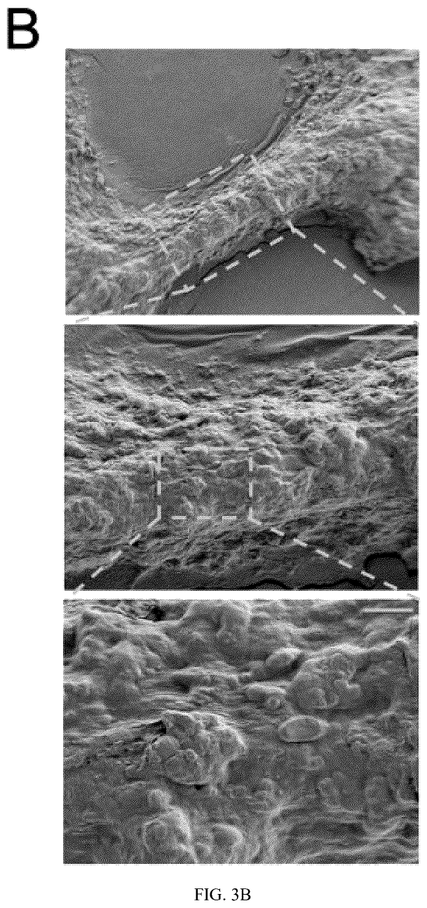

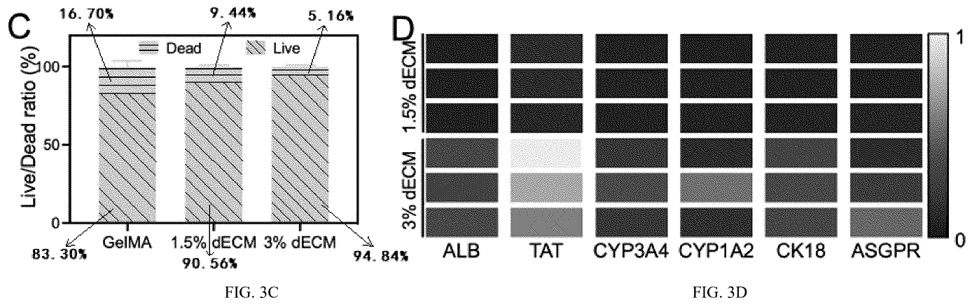

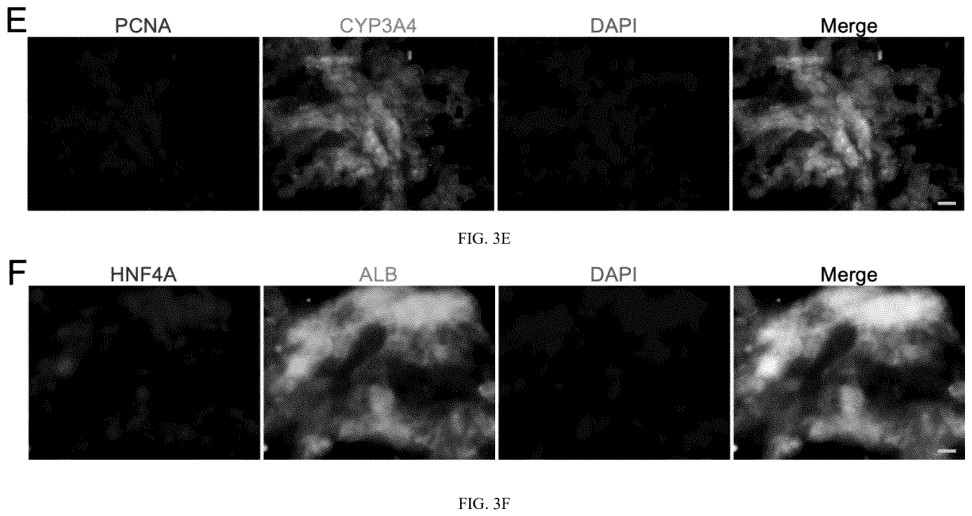

To evaluate the biocompatibility of the dECM based hydrogels, iPSC-heps were selected as the cell source and seeded on the scaffolds printed with a DelMA and 1.5% dECM hydrogel and a DelMA and 3% dECM hydrogel respectively. Cell viability was evaluated by staining live/dead cells on days 1, 3, 5, and 7 after seeding (FIG. 3A). The results indicated that over time, the number and density of cells significantly increased, indicating that all scaffold types were conducive to cell adhesion and growth. Quantitative analysis of live/dead cells on day 7 found that the 3% dECM hydrogel has the highest cell survival rate, indicating a more favorable cell survival environment (FIG. 3C). To further evaluate the effect of high-concentration dECM hydrogels on maintaining cell function, albumin (ALB) secretion, CYP450 family expression, and mRNA levels of key liver transcription factors and functional genes in cells cultured on the 1.5% and 3% dECM hydrogel scaffolds were detected (FIG. 3D). The results showed that a concentration of 3% dECM could significantly enhance the expression of the cell function. Therefore, the 3% dECM based hydrogel was selected for subsequent experiments. To visually evaluate the cell adhesion to the scaffold, the scaffold prepared from the 3% dECM hydrogel was observed under a scanning electron microscope (FIG. 3B). As the magnification of the electron microscope gradually increased, it was evident that cells exhibited a high affinity for the surface of the scaffold. The expression of cell function on the 3% dECM hydrogel scaffold was detected by fluorescence staining. Positive PCNA was found, indicating that the cells had the ability to proliferate, and CYP3A4, ALB and HNF4A staining confirmed the ability of the scaffold to maintain the albumin secretion, metabolic function and biological activity of hepatic cells (FIGS. 3E-3F). Therefore, it was believed that the 3% dECM based hydrogel scaffold was suitable for in vivo liver regeneration applications.

### Embodiment 5

### Efficacy Evaluation of Transplantation With Decm Based Hydrogel Scaffold in Treatment of ALF Mice

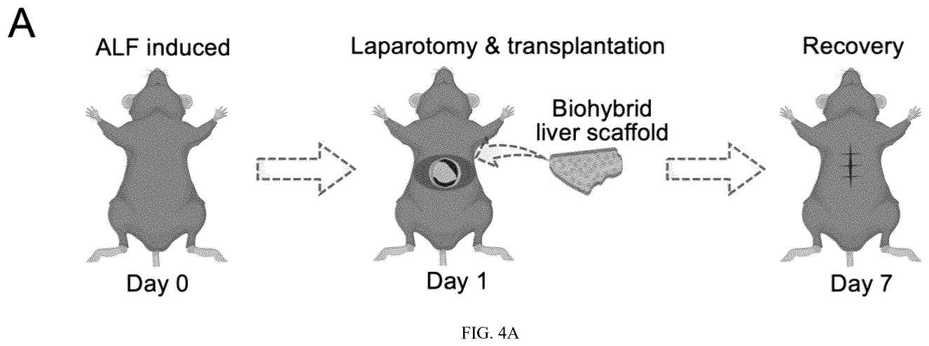

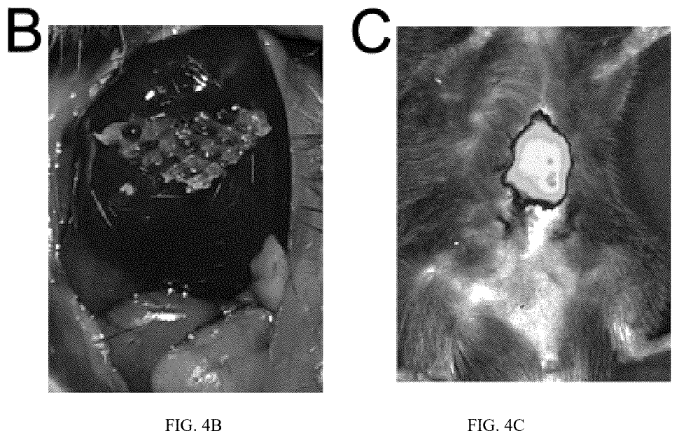

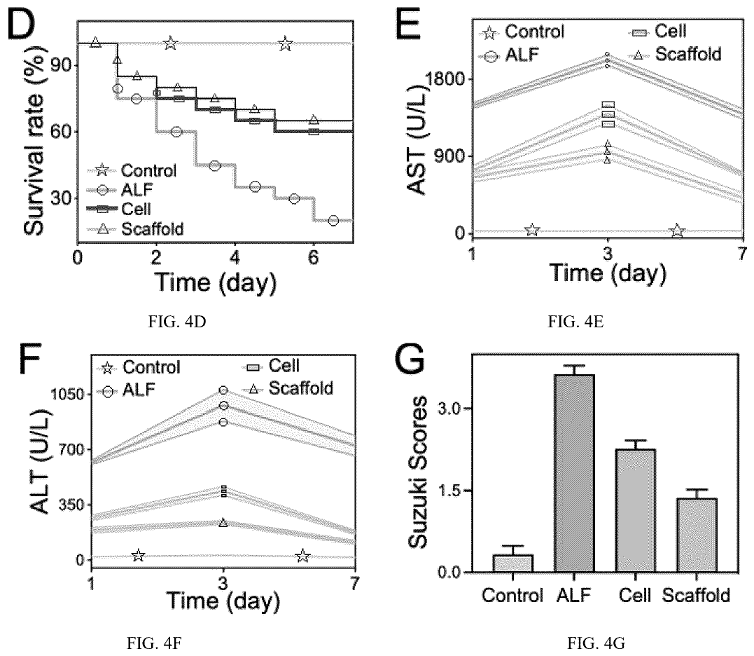

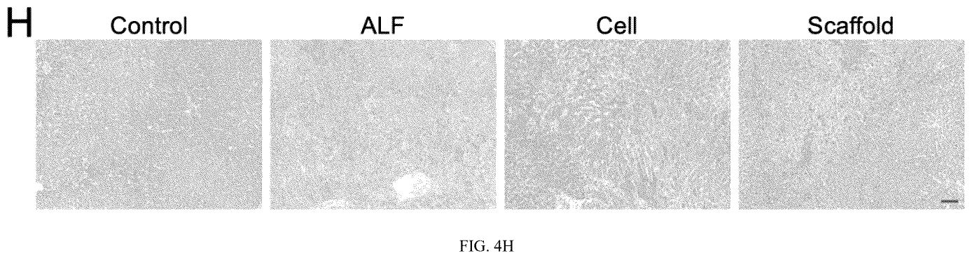

Acute liver failure (ALF) was induced in mice by intraperitoneal injection of D-galactose (D-Gal), the liver was exposed by 1 cm incision in the upper abdomen of mice, and then the hydrogel scaffold was transplanted in situ to evaluate the efficacy in treating acute liver failure (FIGS. 4A-4B). To study the colonization of the hydrogel scaffold in the liver, a fluorescent cell membrane dye 1,1′-Dioctadecyl-3,3,3′,3′-tetramethylindocarbocyanine perchlorate (DIR) was used to stain the hydrogel scaffold before transplantation. After transplantation, a small animal was subjected to in vivo imaging, and as shown in FIG. 4C, the hydrogel scaffold showed obvious high fluorescence intensity in the liver area 7 days after transplantation, and did not migrate to other parts of the body.

To further understand the efficacy of the dECM based hydrogel in the treatment of ALF, the survival rate, liver function and other indicators of mice in each group were detected. The detection indicators include aspartate aminotransferase (AST), which evaluates the degree of hepatic cell damage, and alanine aminotransferase (ALT), which evaluates the degree of hepatic cell damage.

The experiment was conducted in four groups, including

Control group: normal mice;

ALF group: mice induced by acute liver failure;

Cell group: acute liver failure induced mice treated with a simple iPSC-heps cell suspension; and

Scaffold group: acute liver failure induced mice treated with the dECM based microfluidic 3D printing hydrogel prepared in Embodiment 1 of the present disclosure.

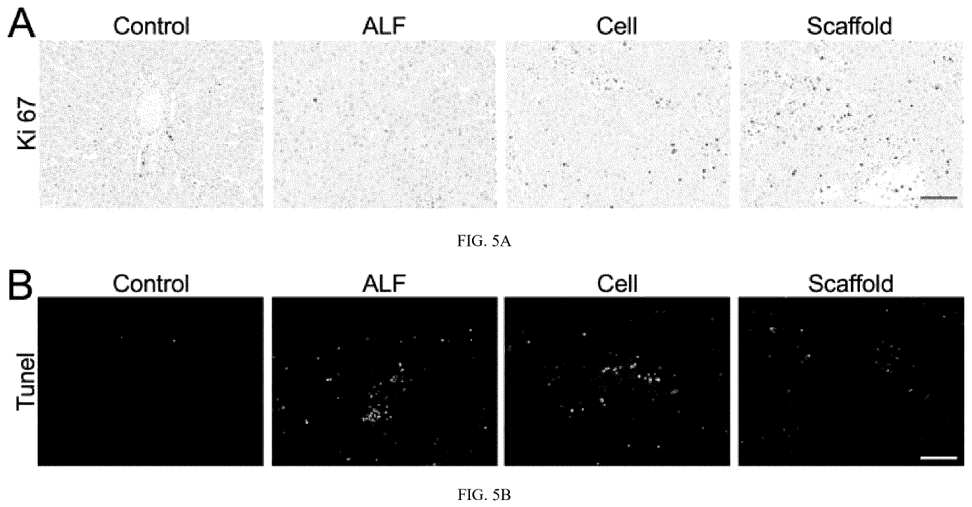

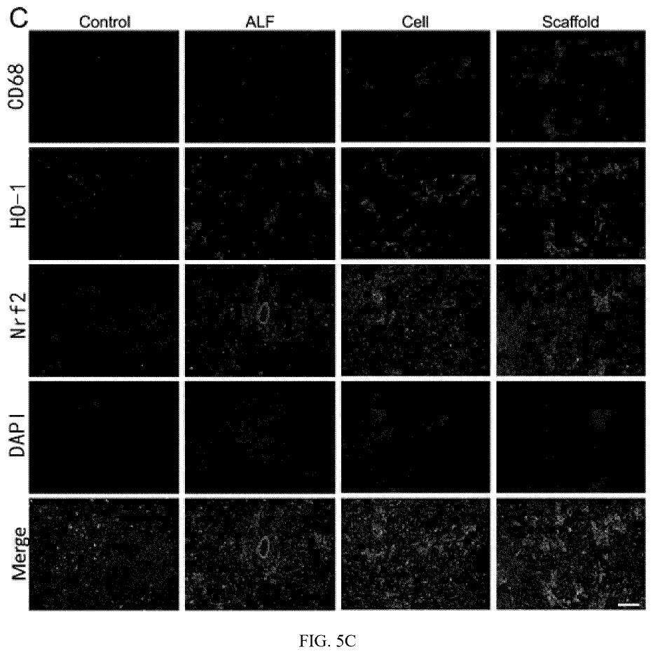

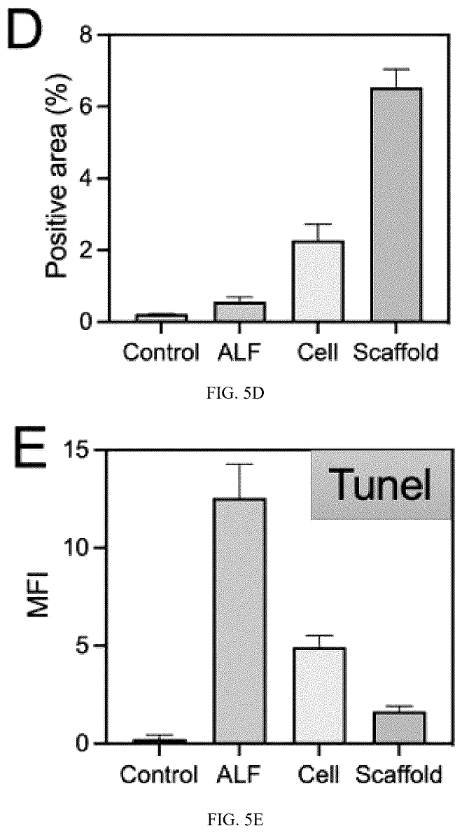

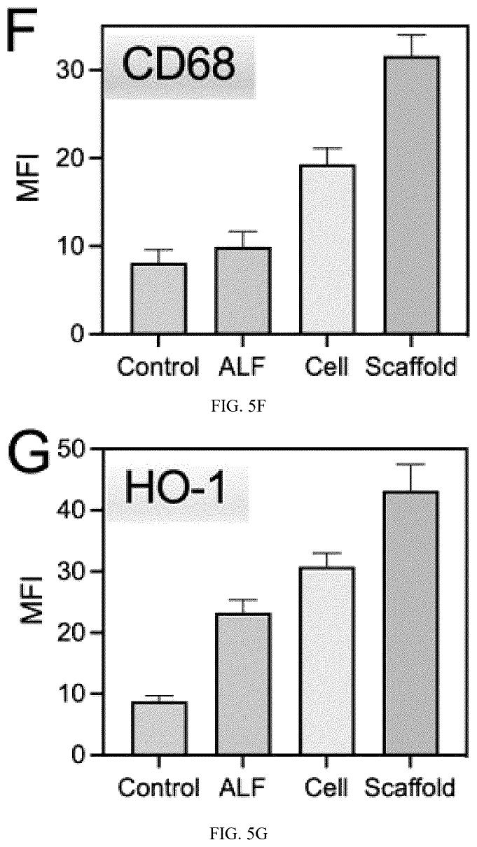

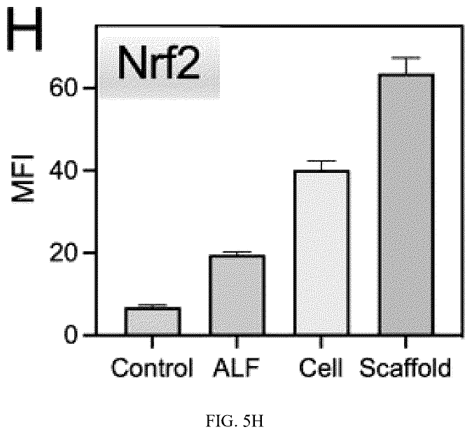

7-day survival monitoring showed that the mortality rates of the Cell group and the Scaffold group were lower than that of the ALF group, with the Scaffold group having a higher survival rate (FIG. 4D). In addition, markers of alanine aminotransferase (ALT) and aspartate aminotransferase (AST) indicated that hepatic cell damage was more effectively controlled and liver function recovery was promoted in the Scaffold group (FIGS. 4E-4F). H&E staining of hepatic tissues showed extensive necrosis in the ALF group, while the necrotic areas were significantly reduced in both the cell treatment groups. Especially in the Scaffold group, the liver necrosis was the smallest, and the Suzuki score was the lowest (FIGS. 4G-4H). This indicated that hepatic injury was restored after scaffold transplantation. In addition, Ki-67 staining was used to evaluate the proliferation of the hepatic cells after transplantation. Compared with the ALF group, both the Cell group and the Scaffold group significantly improved liver cell proliferation, with the Scaffold group having a more pronounced effect on cell proliferation (FIG. 5A, FIG. 5D). Similarly, TUNEL staining showed a significant decrease in the apoptosis levels of the hepatic cells in the Cell group and the Scaffold group, with fewer apoptotic cells in the Scaffold group (FIG. 5B, FIG. 5E). Considering that inflammatory damage and oxidative stress are also major factors affecting the liver regeneration, to find out the specific role of the Scaffold group in liver repair, CD68, HO-1, and Nrf2 staining was performed on the hepatic tissues (FIG. 5C, FIGS. 5F-5H). The results showed that compared with the ALF group and the Cell group, the expression of CD68 was higher in the Scaffold group, indicating larger macrophage aggregation and resistance to inflammatory response. In addition, the activation of the Nrf2/HO-1 antioxidant pathway in the Scaffold group also indicated that the Scaffold group could promote liver recovery by enhancing antioxidant capacity. Therefore, it is believed that the transplantation treatment in the Scaffold group may promote liver repair through anti-inflammatory and antioxidant effects.

The above are only preferred embodiments of the invention and are not used to limit the invention, any modification, equivalent replacement, improvement, etc. made within the spirit and principle of the invention shall fall within the scope of the invention.

What is claimed is:

1. A fish liver decellularized extracellular matrix based microfluidic 3D printing hydrogel, wherein a fish liver decellularized extracellular matrix is combined with gelatin methacryloyl as a scaffold, hepatic spheroids derived from human induced pluripotent stem cells are used as a cell source, and a microfluidic 3D printing technology is used to prepare a mixed material hydrogel scaffold.

2. The fish liver decellularized extracellular matrix based microfluidic 3D printing hydrogel according to claim 1, wherein preparation steps are as follows:
1) preparing the hepatic spheroids derived from the human induced pluripotent stem cells;
2) preparing the fish liver decellularized extracellular matrix; and
3) designing a 3D scaffold model; fully mixing the gelatin methacryloyl, the fish liver decellularized extracellular matrix prepared in the step 2), the hepatic spheroids derived from the human induced pluripotent stem cells prepared in the step 1), and a photoinitiator in a proportion in water to form a biological link; and solidifying the biological link under an ultra violet (UV) irradiation into the scaffold with a fixed shape.

3. The fish liver decellularized extracellular matrix based microfluidic 3D printing hydrogel according to claim 2, wherein the step 2) comprises steps as follows:
a, extracting a liver and intact blood vessels from a fresh fish;
b, continuously perfusing the liver with a surfactant solution at a room temperature for a decellularization to obtain a decellularized material;
c, washing the decellularized material obtained from the step b with a phosphate buffered saline to obtain the fish liver decellularized extracellular matrix; and
d, dissolving the fish liver decellularized extracellular matrix obtained from the step c in an acetic acid aqueous solution containing pepsin.

4. The fish liver decellularized extracellular matrix based microfluidic 3D printing hydrogel according to claim 2, wherein the photoinitiator is 2-hydroxy-2-methyl-1phenyl-1acetone.

5. The fish liver decellularized extracellular matrix based microfluidic 3D printing hydrogel according to claim 3, wherein a surfactant in the surfactant solution is sodium dodecyl sulfonate or sodium deoxycholate.

6. The fish liver decellularized extracellular matrix based microfluidic 3D printing hydrogel according to claim 3, wherein a concentration of the surfactant solution used in the step b is 1%.

7. The fish liver decellularized extracellular matrix based microfluidic 3D printing hydrogel according to claim 3, wherein a continuous perfusion rate for the liver is 10 mL/min, and a perfusion time is 3 h in the step b.

8. The fish liver decellularized extracellular matrix based microfluidic 3D printing hydrogel according to claim 3, wherein in the acetic acid aqueous solution containing the pepsin in the step d, a concentration of the pepsin is 1%, and a concentration of acetic acid is 100 mM.

9. The fish liver decellularized extracellular matrix based microfluidic 3D printing hydrogel according to claim 2, wherein a mass ratio of the fish liver decellularized extracellular matrix to the gelatin methacryloyl to the photoinitiator in a reaction system is (1.5-3):7.5:1.
